(19) United States
(12) Patent Application Publication
(10) Pub. No.: US 2010/0298858 A1
(43) Pub. Date: Nov. 25, 2010

(54) METHODS AND APPARATUS FOR EXTERNAL BEAM RADIATION TREATMENTS OF RESECTION CAVITIES

(76) Inventors: Steven C. Dimmer, Bellevue, WA (US); Fergus Quigley, Seattle, WA (US); Eric D. Nielson, Bellevue, WA (US)

Correspondence Address: PERKINS COIE LLP;PATENT-SEA, P.O. BOX 1247, SEATTLE, WA, 98111-1247, US

(21) Appl. No.: 12/665,498

(22) PCT Filed: Jun. 19, 2008
(86) PCT No.: PCT/US08/67551

§ 371 Date: Jul. 27, 2010

Related U.S. Application Data
(60) Provisional application No. 60/945,042, filed on Jun. 19, 2007.

(51) Int. Cl. A61M 29/00

(52) U.S. Cl. 606/192

(57) ABSTRACT

Apparatus and methods for external beam radiation treatments of resection cavities are described. One embodiment of such an apparatus comprises a stabilization device having an expandable member configured to (a) be implanted in a patient and (b) move from a first position for insertion into a resection cavity within the patient to a second position for stabilizing tissue of the cavity. The apparatus further includes an active marker coupled to the stabilization device and an electrically conductive line connected to the active marker. The active marker is configured to transmit a signal. The electrically conductive line has an internal portion coupled to the stabilization device and an external portion configured to be coupled to a signal generator and/or a signal processor located outside of the patient.

### CROSS-REFERENCE TO RELATED APPLICATION

This application claims benefit of U.S. Patent Application No. 60/945,042, filed Jun. 19, 2007, titled “METHODS AND APPARATUS FOR EXTERNAL BEAM RADIATION TREATMENTS OF RESECTION CAVITIES” which is hereby incorporated by reference in its entirety.

### TECHNICAL FIELD

The present invention is related to radiation oncology and, more specifically, to accurately determining the location of a target for delivering external radiation.

### BACKGROUND

Cancer begins in the cells of the patient and forms malignant tumors that are often treated by surgical resection. Such surgical treatments attempt to remove as much of a tumor as possible, but cancerous cells infiltrate into the tissue adjacent the tumor such that there is no clear boundary. Also, certain procedures seek to limit the treatment margin around the tumor to reduce the amount of healthy tissue removed from the patient. In breast cancer, for example, patients prefer to limit the size of the lumpectomy resection to avoid excessive reduction or non-uniformities of the breast. Both of these factors limit the efficacy of surgical procedures for treating cancer. As such, radiation therapy has become a significant and highly successful process for treating breast cancer, lung cancer, brain cancer and many other types of localized cancers. Radiation therapy is particularly useful for treating (a) tissue after resecting a tumor, (b) centrally located tumors, and/or (c) small cell tumors that cannot be surgically resected. Radiation therapy can also be used as a palliative treatment when a cure is not possible.

Breast cancer has recently been treated by surgically resecting cancerous breast tissue and subsequently treating the remaining tissue surrounding the resection cavity using radiation. Proxima Corporation and Xoft, Inc. have developed breast brachytherapy devices and systems for selectively irradiating the portion of the tissue surrounding the resection cavity created by a lumpectomy. The existing breast brachytherapy devices have a balloon configured to be implanted in the cavity within the breast and an internal radiation source that can be placed within the balloon. After performing a lumpectomy, the balloon is inserted into the surgical cavity and inflated until the balloon presses against the tissue. The balloon is typically left in the patient for approximately five days over which two radiation treatments per day are performed. Each radiation treatment includes inserting the radiation source into the balloon and activating the radiation source to deliver ionizing radiation for approximately 10-15 minutes. After all of the radiation treatments have been performed during the multi-day course of treatment, the balloon is deflated and removed from the patient.

Breast brachytherpy procedures, however, can be challenging. For example, it may be difficult to determine whether the balloon has been inflated accurately and to monitor the balloon to ensure that the balloon has maintained the desired size throughout the multi-day course of treatment. The size of the balloon is currently determined by instilling radiopaque contrast into the balloon and measuring a resulting CT or X-ray image using a ruler. The patient must accordingly undergo a CT scan or another type of X-ray to obtain the image, and then a practitioner must evaluate the image to determine if the balloon is at the desired size. This is time-consuming and expensive, and it should be performed each day during the course of treatment. This process also exposes the patient to additional radiation.

Breast brachytherapy may also have disadvantages associated with using an internal radiation source. For example, the balloon may move within the lumpectomy cavity over the course of treatment, which can cause the internal radiation source to over irradiate some areas and under irradiate other areas. Many existing systems do not detect the relative position between the balloon and the breast to mitigate this problem. Moreover, when the radiation source is asymmetrically positioned within the balloon (e.g., spaced apart from a rotational center line of the balloon), the rotational orientation of the balloon within the lumpectomy cavity can cause the radiation source to be located at an undesirable position relative to the tissue. Conventional techniques also do not identify the rotational orientation of the balloon. This can be problematic because the balloon can move after it has been implanted over the course of treatment, or the balloon may not inflate as planned. Conventional breast brachytherapy systems are also relatively large because they must contain both a balloon and an internal radiation source. Many patients are not comfortable with having a radiation source within their body or with having a large catheter projecting from their body for a number of days, and therefore a sizable number of patients elect not to undergo breast brachytherapy.

In light of the challenges associated with breast brachytherapy procedures, partial breast irradiation using an external radiation beam has been proposed. Although radiation beams, such as Three-Dimensional Conformal Radiation Therapy beams, can shape radiation beams to conform to the target tissue, it is still difficult to use external beam radiation to treat the tissue around the resection cavities in many applications. For example, the size and shape of the cavity may change over the multi-day period typically required for external beam radiation treatments, or the treatment target may move during the treatment sessions. As such, there is a need for improving external beam radiation for partial breast irradiation and other procedures that seek to irradiate controlled treatment margins around resection cavities.

### BRIEF DESCRIPTION OF THE DRAWINGS

FIG. 1 is a side view illustrating an implementation of an apparatus for performing partial breast irradiation.

FIGS. 2A and 2B are side views of apparatus for guided radiation therapy in accordance with two different embodiments.

FIG. 3 is a schematic illustration of a system with an apparatus for guided radiation therapy in accordance with an embodiment.

FIG. 4 is a side view of an apparatus for guided radiation therapy in accordance with another embodiment.

FIG. 5 is a side view of an apparatus for guided radiation therapy in accordance with yet another embodiment.

FIG. 6 is a side view of an apparatus for guided radiation therapy in accordance with still another embodiment.

### DETAILED DESCRIPTION

The following description provides specific details of several embodiments in the context of partial breast irradiation using external beam radiation. However, one skilled in the relevant art will recognize that the invention may be practiced in other contexts and without one or more of these specific details, or with other methods, components, materials, etc. For instance, inflatable devices for temporary or permanent implantation in a patient can have one or more markers as described below for use in external beam radiation therapy procedures described in U.S. patent application Ser. Nos. 11/165,843, filed on 24 Jun. 2005, and 11/166,801, filed on 24 Jun. 2005, both of which are incorporated herein by reference. In other instances, well-known structures associated with target locating and tracking systems have not been shown or described in detail to avoid unnecessarily obscuring descriptions of the embodiments of the invention.

### Overview

Several embodiments are directed toward apparatus for use with localization systems to provide guided external beam radiation therapies. One embodiment of such an apparatus comprises a stabilization device having an expandable member configured to (a) be implanted in a patient and (b) move from a first position for insertion into a resection cavity within the patient to a second position for stabilizing tissue of the cavity. The apparatus further includes an active marker coupled to the stabilization device and an electrically conductive line connected to the active marker. The active marker is configured to transmit a signal. The electrically conductive line has an internal portion coupled to the stabilization device and an external portion configured to be coupled to a signal generator and/or a signal processor located outside of the patient.

Another embodiment of an apparatus for guided radiation therapy comprises an expandable member configured to move from a first position for insertion into a resection cavity within a patient to a second position for stabilizing tissue of a cavity. The apparatus further includes an active marker configured to be implanted in tissue of the patient apart from the expandable member. The active marker is configured to transmit a non-ionizing wireless signal.

Still another embodiment of an apparatus for guided radiation therapy comprises a tissue stabilizing device having an expandable member configured to (a) be implanted in a patient and (b) move from a first position for insertion into a resection cavity within a patient to a second position for stabilizing or otherwise shaping tissue of the cavity. The apparatus further includes an active marker configured to be positioned within the patient relative to the expandable member and a line attached to the active marker. The line has an internal portion configured to be implanted in the patient and an external portion configured to extend externally from the patient.

The invention further includes methods for treating a patient after a procedure that leaves a lumpectomy resection cavity. One embodiment of such a method comprises inserting a distal end of a tissue stabilizing device into the resection cavity and moving an expandable member at the distal end of the tissue stabilizing device from a collapsed configuration to an expanded configuration within the resection cavity. The method further includes localizing an active marker positioned within the patient relative to the expandable member by transmitting a non-ionizing location signal from the active marker and calculating a position of the active marker in an external coordinate system based on the location signal. The active marker is attached to a line having an internal portion configured to be implanted in the patient and an external portion configured to extend outside of the patient.

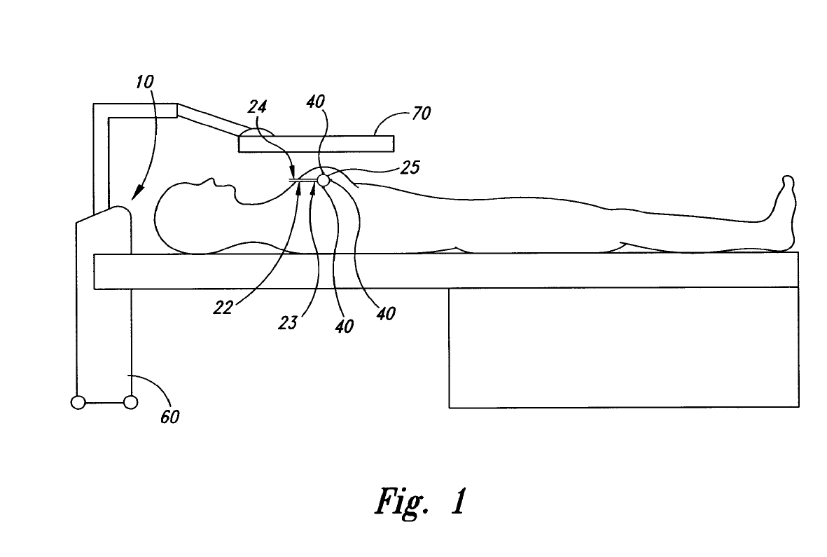

### Embodiments of Apparatus for Facilitating Radiation Treatment FIG. 1 is a side view of a localization system 10 with an apparatus 20 for facilitating radiation treatment of a target in accordance with an embodiment of the invention. The apparatus 20 includes a tube 22 having a first end 23 configured to be inserted into the patient and a second end 24 configured to be outside of the patient. The tube 22 can be a catheter, such as a multilumen silicon catheter, or other type of device that can be percutaneously inserted into the breast or other part of the body. The apparatus 20 further includes an expandable member 25 at the first end 23 of the tube 22. The expandable member 25 can be a balloon or other type of device configured to create and hold a desired shape. For example, the expandable member 25 can be an inflatable bladder, mechanical linkage, a mesh of shape memory material, or other suitable devices that can be inserted into a patient and moved between a collapsed configuration and an enlarged or expanded configuration. When the expandable member 25 is a balloon, it is typically inserted into the patient in a collapsed configuration (not shown) and then filled until the balloon expands to a desired diameter.

The apparatus 20 further includes a plurality of markers 40 associated with the expandable member 25. One or more of the markers 40, for example, can be configured to move with the expandable member 25 to an expanded orientation. The markers 40 can comprise wired sensors configured to transmit independent location signals in response to an energy source that is external to the body, and/or one or more of the markers 40 can comprise wired transmitters that transmit source energy to a sensor array that is external to the body. For example, the active markers 40 can be single coil or multiple coil sensors that produce an electrical current in response to the strength of an externally supplied alternating magnetic field. Alternatively, one or more of the active markers can be a wireless active sensor that wirelessly transmits location signals in response to wirelessly transmitted excitation signals. Such wireless active markers can comprise a casing and a magnetic transponder in the casing as described in U.S. patent application Ser. Nos. 11/243,478 and 11/166,801, both of which are incorporated herein by reference in their entirety.

In several embodiments, one or more markers 40 are attached to or otherwise embedded in the expandable member 25 such that the markers move in direct correspondence to the movement of the expandable member 25. In other embodiments, markers 40 can be attached to a sheath or a mesh that surrounds the expandable member 25 so that expansion of the expandable member 25 causes a corresponding expansion of the sheath or mesh. One or more markers 40 can also be attached to the tube 22. The markers 40 are accordingly associated with the expandable member 25 such that the markers 40 move based on the position, rotation, and/or expansion-contraction of the expandable member 25. In the illustrated embodiment shown in FIG. 1, more specifically, three markers 40 are associated with the expandable member 25 of the apparatus 20. In other applications, a single marker, two markers, or more than three markers can be used depending upon the particular application. Two markers, for example, are highly desirable because the target can be located accurately, and also because relative displacement between the markers over time can be used to monitor the status and position of the expandable member 25 in the patient 6.

The localization system 10 can determine the location of the active markers 40 in real-time to facilitate external beam radiation therapy for partial breast irradiation or other therapies. The localization system 10 can include a controller 60 and a field device 70. The field device 70 can generate one or more alternating magnetic fields that the active markers 40 sense, or the field device 70 can sense one or more alternating magnetic fields generated by the active markers 40. In either case, the controller 60 receives location signals from either the field device 70 or the active markers 40 and determines the actual location of the individual markers 40 in a three-dimensional reference frame when the markers are within or on the patient 6. In a particular embodiment of the system illustrated in FIG. 1, the localization system 10 tracks the three-dimensional coordinates of the markers 40 in real time to an absolute external reference frame during the setup process and while irradiating the patient to mitigate collateral effects on adjacent healthy tissue and to ensure that the desired dosage is applied to the target tissue.

Several embodiments of the apparatus 20 enable accurate determination of the size of the expandable member within the breast without taking expensive CT images and manually assessing the images. This aspect is very useful because the diameter or size of the expandable member 25 may initially define the shape of the resection cavity, but the expandable member 25 may change over the course of the treatment. For example, the expandable member 25 may collapse or have a slow leak such that the size and shape of the expandable member may change over the multi-day treatment course. This could result in a change in the shape of the resection cavity, which could cause the external beam radiation to irradiate healthy tissue but miss targeted tissue. By localizing the relative positions of the markers 40, changes in the size and shape of the expandable member 25 can be determined before, during, and after each treatment session to ensure that the desired dose of radiation is accurately delivered to the correct tissue.

Several embodiments of the apparatus 20 can also track movement of the treatment target throughout the course of therapy to accurately deliver external beam radiation within the treatment margin. Breast tissue is soft and pliable such that it may be difficult to hold the treatment target at the isocenter of the external radiation beam. The breast is also likely subject to movement caused by thoracic expansion/contraction during respiration. Several embodiments of the apparatus 20 are also useful for detecting movement of the patient or other displacement of the breast in real-time during therapy. As a result, the apparatus 20 is expected to provide accurate measurements to confirm the status and the location of the treatment target throughout the course of therapy.

Several embodiments of the apparatus 20 also track the rotational orientation of the expandable member 25 relative to the breast or the radiation beam throughout the course of treatment. The rotational orientation of the expandable member 25 may be important in several applications because the resection cavity is generally not spherical such that the rotational orientation affects the profile of the treatment margin relative to the position of the external beam. The markers 40 can be tracked or otherwise located using the localization system 10 to determine rotational orientation of the resection cavity relative to the external beam

### 1. General Operation of Selected Markers and Localization Systems

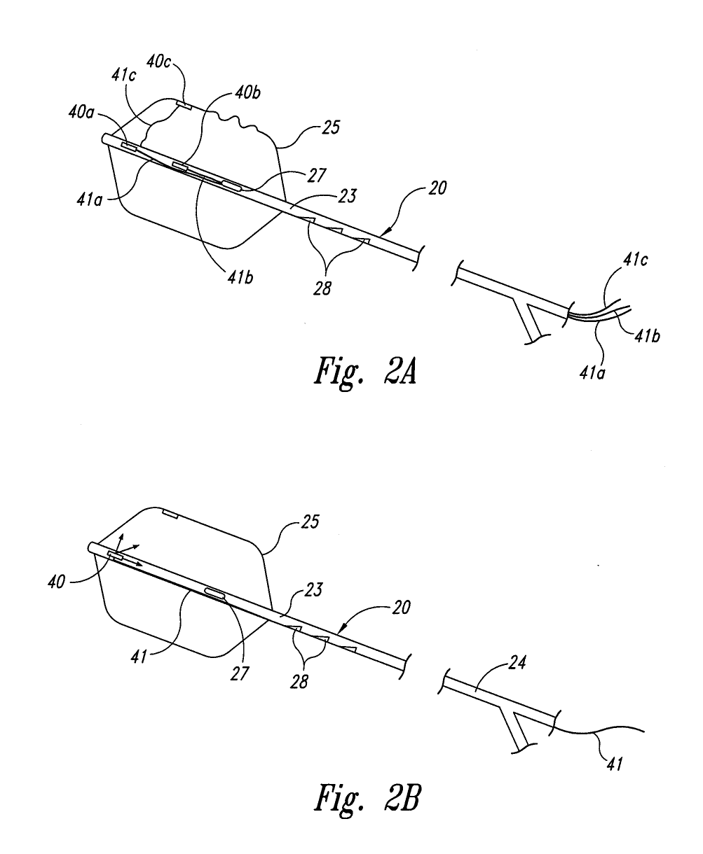

FIG. 2A is a side view illustrating a specific embodiment of the apparatus 20 in more detail. In this embodiment, the apparatus 20 has a first marker 40a at a first location along the tube 22, a second marker 40b spaced apart from the first marker 40a at a second location along the tube 22, and a third marker 40c carried by the expandable member 25. The markers 40a-c can be individual coils that sense and/or generate an alternating magnetic field depending on whether the field device 70 is a field generator or a field sensor. The individual markers 40a-c, for example, can be tuned to resonate at unique frequencies relative to each other, or the markers 40a-c can receive and respond to a common frequency. The apparatus 20 also includes conductive lines 41a-c electrically coupled to the markers 40a-c, respectively. Individual conductive lines can have an internal portion attached to a corresponding marker and an external portion configured to extend externally of the patient and be attached to the controller 60 (FIG. 1). As such, the conductive lines 41a-c carry signals between the markers 40a-c and the controller 60 (FIG. 1).

The expandable member 25 of the apparatus 20 shown in FIG. 2A is a balloon or other inflatable device. The balloon, for example, can be a flexible elastic material or a flexible inelastic material depending on the application. In one embodiment, the balloon is made from materials such as polyethylene terephthalate (PET) or nylon. The apparatus 20 accordingly also has first port 27 along the tube 22 within the balloon for injecting or draining saline solution or other biocompatible fluids to inflate/deflate the balloon. The apparatus 20 can also include one or more drains 28 along the tube outside of the expandable member 25.

FIG. 2B is a side view illustrating another specific embodiment of the apparatus that is similar to the apparatus shown in FIG. 2A. In the apparatus 20 shown in FIG. 2B, however, the marker 40 is a multi-coil marker that senses or transmits signals along separate axes. For example, the marker 40 in FIG. 2B can have three coils arranged along three different axes, such as three orthogonal axes. Although the apparatus 20 of FIG. 2B is shown with only a single marker 40, it can have two or more multi-axis (e.g., multi-coil) active markers carried by the expandable member 25 and/or the tube 22.

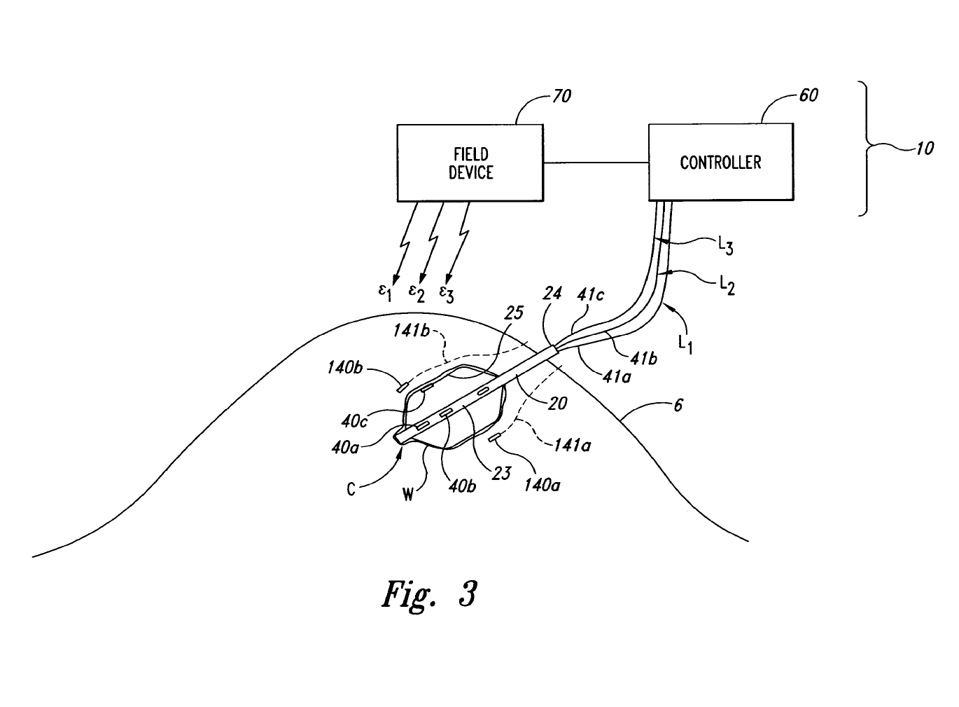

FIG. 3 is a schematic view illustrating the operation of an embodiment of the localization system 10 and markers 40a-c for treating a target in the breast of the patient. The markers 40a-c are used to determine the size, location and/or rotational orientation of the expandable member 25 and/or the wall W of a resection cavity C in which the expandable member 25 is positioned. More specifically, the localization system 10 determines the locations of the markers 40a-c and provides objective target position data to a memory, user interface, linear accelerator and/or other device in real time during setup, treatment, deployment, simulation, surgery, and/or other medical procedures. In one embodiment of the localization system, real time means that indicia of objective coordinates are provided to a user interface at (a) a sufficiently high refresh rate (i.e., frequency) such that pauses in the data are not humanly discernable and (b) a sufficiently low latency to be at least substantially contemporaneous with the measurement of the original signal. In other embodiments, real time is defined by higher frequency ranges and lower latency ranges for providing the objective data, or in still other embodiments, real time is defined as providing objective data responsive to the location of the markers (e.g., at a periodicity or frequency that adequately tracks the location of the target in real time and/or at a latency that is at least substantially contemporaneous with obtaining position data of the markers).

In an embodiment of the system 10 shown in FIG. 3, the field device 70 is an excitation source 70 (e.g., a pulsed magnetic field generator) that generates an excitation energy to energize at least one of the markers 40a-c in the patient 6. The excitation source 70 shown in FIG. 3 can produce a pulsed magnetic field at one or more frequencies. In one embodiment, the excitation source 70 generates an alternating magnetic field at a frequency and the markers 40a-c generate individual location signals L₁₋₃ that are transmitted to the controller via conductive lines 41a-c, respectively. In another embodiment, the excitation source 70 can frequency multiplex the magnetic field at a first frequency E₁ to energize only the first marker 40a, a second frequency E₂ to energize only the second marker 40b, and a third frequency E₃ to energize only the third marker 40c. In response to the three excitation energies, the markers 40a-c generate location signals L₁₋₃, respectively, at unique response frequencies. The location signals L₁₋₃ generated by the markers 40a-c at the unique frequencies are also transmitted to the controller 60 via conductive lines 41a-c, respectively.

The computer-operable media in the controller 60, or in a separate signal processor, also includes instructions to determine the absolute positions of each of the markers 40a-c in a three-dimensional reference frame. Based on signals provided via the conductive lines 41a-c corresponding to the magnitude of each of the location signals L₁₋₃, the controller 60 and/or a separate signal processor calculates the absolute coordinates of each of the markers 40a-c in the three-dimensional reference frame.

In another embodiment of the system 10, the field device 70 is an array of sensors and the markers 40a-c generate individual magnetic fields. The markers 40a-c of this embodiment, for example, can generate individual excitation energies and the field device 70 senses the individual excitation energies and sends location signals corresponding to the separate markers 40a-c to the controller 60. In one particular embodiment, the first marker 40a can generate a first excitation energy at a first frequency, the second marker 40b can generate a second excitation energy at a second frequency, and the third marker 40c can generate a third excitation energy at a third frequency. The individual excitation energies from the markers 40a-c can be time multiplexed. Based on signals provided via the field device 70 of this embodiment, the controller 60 and/or a separate signal processor can calculate the absolute coordinates of each of the markers 40a-c in the Three-dimensional reference frame.

Another embodiment illustrated in FIG. 3 includes one or more additional markers 140a-b implanted in tissue apart from the expandable member 25. The markers 140a-b can be leadless markers that produce wirelessly transmitted location signals in response to wirelessly transmitted excitation energy. Suitable leadless active markers are described in U.S. patent application Ser. No. 11/166,801 incorporated by reference above. In another embodiment, the active markers 140a-b can be connected to lead wires 141a-b, respectively. The lead wires 141a-b can accordingly include internal portions connected to the corresponding active markers 140a-b and external portions projecting outside of the patient. When the active markers 140a-b are connected to the lead wires 141a-b, the active markers 140a-b can be sensors that receive excitation energy from the field device 70 or generate the excitation energy to be sensed by the field device 70.

The embodiments of apparatus for guided radiation therapy described above can be used in methods for treating a patient after a procedure that leaves a resection cavity within the patient. An embodiment of such a method comprises inserting a distal end of a tissue stabilization device into the resection cavity and moving an expandable member at the distal end of the tissue stabilization device from a collapsed configuration to an expanded configuration within the resection cavity. The expandable member at least partially shapes the resection cavity in the expanded configuration. The method can further include localizing an active marker positioned within the patient relative to the expandable member by transmitting a non-ionizing location signal from the active marker and calculating a position of the active marker in an external coordinate system based on the location signal. The active marker can be attached to a line having an internal portion configured to be implanted in the patient and an external portion configured to extend outside of the patient.

Additional embodiments of the method can further comprise directing an external radiation beam toward the resection cavity based on the calculated position of the active marker(s) and/or repeating the inserting, moving and localizing procedures over a period of a plurality of days without removing the expandable member from the resection cavity. For example, the external radiation beam can irradiate the resection cavity based on the calculated position of the marker(s) during treatment fractions that occur over a plurality of days (e.g., usually about 3-7 days for partial breast irradiation treatments). The expandable member can remain in the expanded configuration throughout the plurality of days of the treatment program. Additionally, after the radiation fractions have been completed at the end of the treatment program, the method can further include removing the tissue stabilization device from the patient. For example, the tissue stabilization device can be explanted from the patient after the number of days of the treatment program.

### 2. Real time Tracking

The localization system 10 and markers 40 enable real time tracking of the target and/or status of the expandable member 25 relative to an external reference frame outside of the patient during treatment planning, set up, irradiation sessions, and at other times of the radiation therapy process. In many embodiments, real time tracking means collecting position data of the markers, determining the locations of the markers in an external reference frame (i.e., a reference frame outside the patient), and providing an objective output in the external reference frame responsive to the location of the markers. The objective output is provided at a frequency/periodicity that adequately tracks the target in real time, and/or a latency that is at least substantially contemporaneous with collecting the position data (e.g., within a generally concurrent period of time).

For example, several embodiments of real time tracking are defined as determining the locations of the markers and calculating the locations relative to an external reference frame at (a) a sufficiently high frequency/periodicity so that pauses in representations of the target location at a user interface do not interrupt the procedure or are readily discernable by a human, and (b) a sufficiently low latency to be at least substantially contemporaneous with the measurement of the location signals from the markers. Alternatively, real time means that the location system 10 calculates the absolute position of each individual marker 40 and/or the location of the target at a periodicity of approximately 1 ms to 5 seconds, or in many applications at a periodicity of approximately 10-100 ms, or in some specific applications at a periodicity of approximately 20-50 ms. In applications for user interfaces, for example, the periodicity can be 12.5 ms (i.e., a frequency of 80 Hz), 16.667 ms (60 Hz), 20 ms (50 Hz), and/or 50 ms (20 Hz). Additionally, real time tracking can further mean that the location system 10 provides the absolute locations of the markers 40, the target, and/or the expandable member 25 to a memory device, user interface, linear accelerator, or other device within a latency of 10 ms to seconds from the time the localization signals were transmitted from the markers 40. In more specific applications, the location system generally provides the locations of the markers 40, target, or an instrument within a latency of about 20-50 ms. The location system 10 accordingly provides real time tracking to monitor the position of the markers 40 and/or the target with respect to an external reference frame in a manner that is expected to enhance the efficacy of radiation therapy.

Alternatively, real time tracking can further mean that the location system 10 provides the absolute locations of the markers 40 and/or the target to a memory device, user interface or other device within a latency of 10 ms to 5 seconds from the time the localization signals were transmitted from the markers 40. In more specific applications, the location system generally provides the locations of the markers 40 and/or target within a latency of about 20-50 ms. The location system 10 accordingly provides real time tracking to monitor the position of the markers 40 and/or the target with respect to an external reference frame in a manner that is expected to enhance the efficacy of radiation therapy because higher radiation doses can be applied to the target and collateral effects to healthy tissue can be mitigated.

Alternatively, real-time tracking can further be defined by the tracking error. Measurements of the position of a moving target are subject to motion-induced error, generally referred to as a tracking error. According to specific embodiments, the localization system 10 and at least one marker 4 enable real time tracking of the target or other instrument relative to an external reference frame with a tracking error that is within clinically meaningful limits.

Tracking errors are due to two limitations exhibited by any practical measurement system, specifically (a) latency between the time the target position is sensed and the time the position measurement is made available, and (b) sampling delay due to the periodicity of measurements. For example, if a target is moving at 5 cm/s and a measurement system has a latency of 200 ms, then position measurements will be in error by 1 cm. The error in this example is due to latency alone independent of any other measurement errors, and is simply due to the fact that the target or instrument has moved between the time its position is sensed and the time the position measurement is made available for use. If the measurement system further has a sampling periodicity of 200 ms (i.e., a sampling frequency of 5 Hz), then the peak tracking error increases to 2 cm, with an average tracking error of 1.5 cm.

For a real time tracking system to be useful in medical applications, it is desirable to keep the tracking error within clinically meaningful limits. For example, in a system for tracking motion of a tumor or an instrument for radiation therapy, it may be desirable to keep the tracking error within 5 mm. Acceptable tracking errors may be smaller when tracking other organs for radiation therapy. In accordance with aspects of the present invention, real time tracking refers to measurement of target position and/or rotation with tracking errors that are within clinically meaningful limits.

### 3. Additional Embodiments of Apparatus for Facilitating Radiation Treatment

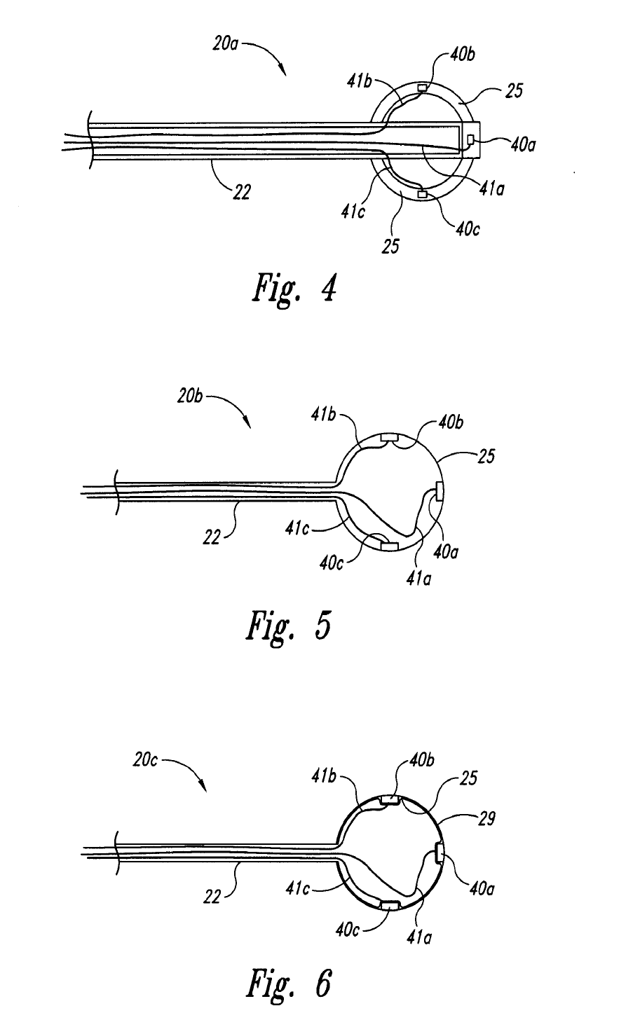

FIGS. 4-6 illustrate additional embodiments of apparatus for facilitating radiation treatment of a target in a patient. FIG. 4, more specifically, illustrates an apparatus 20a that includes the tube 22 and the expandable member 25 at one end of the tube 22. The markers 40 can be embedded within the wall of the expandable member 25 such that the markers 40 move with the expandable member 25 as it is inflated and deflated. The apparatus 20a can include one or more markers 40 embedded within the wall of the expandable member 25, and in optional embodiments one or more markers 40 can also be attached to a distal end of the tube 22.

FIG. 5 is a schematic cross-sectional view illustrating an apparatus 20b in accordance with yet another embodiment. In this embodiment, the apparatus includes the shaft 22 and the expandable member 25 at one end of the shaft. The markers 40 are attached to an interior or exterior surface of the expandable member 25. The markers can be adhered or otherwise attached to the desired surface of the expandable member 25 using an adhesive.

FIG. 6 illustrates another embodiment of an apparatus 20c. In this embodiment, the apparatus 20c includes the tube 22 and the expandable member 25 attached to one end of the tube. This embodiment can further include a flexible member 29 around or otherwise attached to the expandable member 25. The markers 40 are attached to the flexible member 29, and the flexible member 29 can be a sheath or a mesh. In operation, the expandable member 25 presses against the flexible member 29 and expands the flexible member 29 to fill the lumpectomy cavity in the patient. The markers 40 accordingly travel with the movement of the expandable member 25.

From the foregoing, it will be appreciated that specific embodiments of the invention have been described herein for purposes of illustration, but well-known structures and functions have not been shown or described in detail to avoid unnecessarily obscuring the description of the embodiments of the invention. Where the context permits, singular or plural terms may also include the plural or singular term, respectively. Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of features are not precluded. It will also be appreciated that specific embodiments of the invention have been described herein for purposes of illustration, but that various modifications may be made without deviating from the inventions. For example, many of the elements of one of embodiment can be combined with other embodiments in addition to, or in lieu of, the elements of the other embodiments. Accordingly, the invention is not limited except as by the appended claims.

I/We claim:

1. An apparatus for guided radiation therapy, comprising:
a stabilization device having an expandable member configured to (a) be implanted in a patient and (b) move from a first position for insertion into a resection cavity within the patient to a second position for stabilizing tissue of the cavity;
an active marker coupled to the stabilization device, wherein the active marker is configured to transmit a signal; and
an electrically conductive line connected to the active marker, wherein the electrically conductive line has an internal portion coupled to the stabilization device and an external portion configured to be coupled to a signal generator and/or a signal processor located outside of the patient.

2. The apparatus of claim 1 wherein the stabilization device further comprises a catheter and the expandable member comprises a balloon at a distal end of the catheter.

3. The apparatus of claim 1 wherein the expandable member comprises a scaffold.

4. The apparatus of claim 1 wherein the active marker comprises a leadless marker having a core and coil wrapped around the core.

5. The apparatus of claim 1 wherein the stabilization device further comprises a catheter and the active marker is attached to the catheter.

6. The apparatus of claim 1 wherein the active marker is attached to the expandable member.

7. The apparatus of claim 1 wherein the stabilization device further comprises a catheter, the active marker comprises a first active marker attached to the catheter, the electrically conductive line has a first wire coupled to the first active marker and a second wire, and the system further comprises a second active marker attached to the expandable member and coupled to the second wire.

8. The apparatus of claim 1 wherein the active marker comprises three coils arranged orthogonally to each other, and the electrically conductive line comprises three wires, wherein each wire is electrically connected to a single one of the coils.

9. The apparatus of claim 1 wherein the stabilization device further comprises a catheter, the active marker comprises a first active marker attached to the catheter, the electrically conductive line comprises a first wire, a second wire, and a third wire, and the system further comprises a second active marker attached to the catheter and coupled to the second wire, and a third active marker attached to the expandable member and coupled to the third wire.

10. An apparatus for guided radiation therapy, comprising:
an expandable member configured to move from a first position for insertion into a resection cavity within a patient to a second position for stabilizing tissue of the cavity; and
an active marker configured to be implanted in tissue of the patient apart from the expandable member, wherein the active marker is configured to transmit a non-ionizing wireless signal.

11. The apparatus of claim 10 wherein the expandable member comprises a balloon.

12. The apparatus of claim 10 wherein the expandable member comprises a scaffold.

13. The apparatus of claim 10 wherein the active marker comprises a first active marker, and the system further comprises a second active marker configured to be implanted in tissue of the patient apart from the expandable member, and wherein the second active marker is configured to transmit a non-ionizing wireless signal.

14. The apparatus of claim 10 wherein the active marker comprises a leadless marker having a core and a coil wrapped around the core.

15. The apparatus of claim 10 wherein the apparatus further comprises a second active marker coupled to the expandable member and a wire coupled to the second active marker, and wherein the wire has an internal portion configured to be implanted in the patient and an external portion configured to be electrically coupled to a signal generator and/or a signal processor located outside of the patient.

16. The apparatus of claim 10 wherein the active marker comprises a first active marker and the system further comprises a catheter attached to the expandable member and a second active marker attached to the catheter.

17. The apparatus of claim 16 wherein the second active marker comprises a coil and has a wire with an internal portion configured to be implanted in the patient and an external portion configured to be coupled to a signal generator and/or a signal processor located outside of the patient.

18. The apparatus of claim 16 wherein the second active marker comprises a leadless marker having a core and a coil wrapped around the core.

19. An apparatus for guided radiation therapy, comprising:
a tissue stabilizing device having an expandable member configured to (a) be implanted in a patient and (b) move from a first position for insertion into a resection cavity within a patient to a second position for stabilizing tissue of the cavity;
an active marker configured to be positioned within the patient relative to the expandable member; and
a line attached to the active marker, wherein the line has an internal portion configured to be implanted in the patient and an external portion configured to extend externally from the patient.

20. The apparatus of claim 19 wherein the active marker is carried by the stabilizing device and the line comprises an electrically conductive line, and wherein the external portion of the line is configured to be connected to a signal generator and/or a signal processor.

21. The apparatus of claim 19 wherein the active marker comprises a leadless marker configured to be implanted in the patient apart from stabilizing device and transmit a non-ionizing signal, the line comprises a tether attached to the leadless marker, and wherein the leadless marker has a core and a coil wrapped around the core.

22. A method for treating a patient after a procedure that leaves a resection cavity within the patient, the method comprising:
inserting a distal end of a tissue stabilization device into the resection cavity;
moving an expandable member at the distal end of the tissue stabilization device from a collapsed configuration to an expanded configuration within the resection cavity such that the expandable member at least partially shapes the resection cavity; and
localizing an active marker positioned within the patient relative to the expandable member by transmitting a non-ionizing location signal from the active marker and calculating a position of the active marker in an external coordinate system based on the location signal, wherein the active marker is attached to a line having an internal portion configured to be implanted in the patient and an external portion configured to extend outside of the patient.

23. The method of claim 22 wherein the method further comprises directing an external radiation beam toward the resection cavity based on the calculated position of the active marker.

24. The method of claim 22, further comprising localizing a leadless active marker by wirelessly transmitting excitation and localization signals.

25. The method of claim 22 wherein localizing comprises transmitting a wired excitation signal via the line and the active marker.

26. The method of claim 22 wherein localizing comprises transmitting a wireless excitation signal from a field device and transmitting a localization signal from the active marker via the line.

27. The method of claim 22, further comprising implanting a leadless active marker apart from the expandable member

28. The method of claim 22, further comprising implanting a second active marker apart from the expandable member, wherein the second active marker is connected to a conductive line having a distal portion at the second active marker and a proximal portion configured to be coupled to a controller.

29. The method of claim 28, further comprising explanting the second active marker.

30. The method of claim 22 wherein the tissue stabilization device has a plurality of active markers, and the method further comprises determining orientation including pitch, roll and yaw of the resection cavity and/or the expandable member within the resection cavity.

31. The method of claim 22 wherein the expandable member comprises a balloon, and moving from the collapsed position to the expanded position comprises injecting a fluid into the balloon.

32. The method of claim 22 wherein the expandable member comprises a scaffold, and moving from the collapsed position to the expanded position comprises withdrawing a sheath proximally relative to the scaffold.

33. The method of claim 22 wherein the expandable member comprises a scaffold, and moving from the collapsed position to the expanded position comprises urging the scaffold outward.

34. The method of claim 22, further comprising repeating the inserting, moving and localizing procedures over a period of a plurality of days without removing the expandable member from the resection cavity.

35. The method of claim 34 wherein the expandable member is maintained in the expanded configuration throughout the plurality of days.

36. The method of claim 34 wherein the expandable member is maintained in the expanded configuration without being moved into the contracted position for a period of at least three days.

37. The method of claim 34, further comprising removing the tissue stabilization device from the resection cavity after the plurality of days.
